(19) United States
(12) Patent Application Publication
(10) Pub. No.: US 2019/0240306 A1
(43) Pub. Date: Aug. 8, 2019

(54) ANTI-OPIOID VACCINES

(71) Applicant: The Scripps Research Institute, La Jolla, CA (US)

(72) Inventor: Kim D. Janda, La Jolla, CA (US)

(21) Appl. No.: 16/344,229

(22) PCT Filed: Oct. 17, 2017
(86) PCT No.: PCT/US17/56940

§ 371 Date: Apr. 23, 2019

Related U.S. Application Data
(60) Provisional application No. 62/411,850, filed on Oct. 24, 2016.

(51) Int. Cl. A61K 39/00; A61K 39/385; A61P 25/36; C07K 16/44

(52) U.S. Cl. CPC A61K 39/0013; A61K 39/385; A61P 25/36; C07K 16/44; A61K 2039/6012; A61K 2039/55561; A61K 2039/6037; A61K 2039/55544; A61K 2039/55505

(57) ABSTRACT

The invention provides haptens and methods of preparation thereof, for producing immunoconjugates of oxycodone and hydrocodone suitable for raising antibodies in animals specific for the opioid drugs. Administration of the opioid-speeific antibodies to humans having the opioid drugs in a body fluid such as blood serum, binds the opioid drugs to the antibody, making the opioids unavailable for producing a drug effect. Accordingly the antibodies can be used to reverse the effects of a drug overdose of oxycodone and hydrocodone, or to reverse a perceived mental effect of the drugs, such as euphoria.

### CROSS-REFERENCE TO RELATED APPLICATION

This application claims the priority of U.S. provisional application Ser. No. 62/411,850, filed Oct. 24, 2016, the disclosure of which is incorporated by reference herein in its entirety.

### STATEMENT OF GOVERNMENT SUPPORT

This invention was made with government support under 1UH2DA041146-02 awarded by the National Institute on Drug Abuse. The government has certain rights in the invention.

### BACKGROUND

Prescription opioids (POs) such as oxycodone and hydrocodone are highly effective medications for pain management, yet they also present a substantial risk for abuse and addiction. The consumption of POs has been escalating worldwide, resulting in tens of thousands of deaths due to overdose each year. Pharmacokinetic strategies based upon vaccination present an attractive avenue to suppress PO abuse. Herein, the preparation of two active PO vaccines is described that were found to elicit high-affinity anti-opioid antibodies through a structurally-congruent drug-hapten design. Administration of these vaccines resulted in a significant blockade of opioid analgesic activity, along with an unprecedented increase in drug serum half-life. Importantly, the daunting constraints of active vaccination not being able to protect against fatal overdose was refuted.

Prescription opioid (PO) pain relievers form the foundation of modern pain management therapy, yet they are also among the most commonly misused and abused medications (FIG. 1). Indeed, the illicit use of these medicines has reached epidemic levels in the U.S., with 18,893 PO overdose deaths reported in 2014.^{[1]} Notably, these drugs now cause more fatalities than heroin each year.^{[2]} This trend is not isolated to North America—the consumption of POs and PO overdoses have also increased across Europe, especially in the United Kingdom, Germany and Spain.^{[3]} While the misuse of nearly all POs has increased in recent years, oxycodone and hydrocodone products are by far the most widely and commonly abused opioids.^{[1-2, 4]}

Oxycodone and hydrocodone are effective semisynthetic opioid analgesics that predominantly activate mu opioid receptors (MORs) in the central nervous system (CNS). However, the euphoric effects of oxycodone and hydrocodone make them a common source for substance dependence and iatrogenic addiction. Both acute and chronic abuse of these medicines can lead to several serious complications, such as respiratory depression and cerebral hypoxia. The Drug Abuse Warning Network (DAWN) has reported that oxycodone is by far the most common PO associated with emergency department visits (151,218), followed immediately by hydrocodone (82,480).^{[5]} Devastatingly, PO-related fatalities involving oxycodone and hydrocodone overdose increased by nearly 4-fold between 2000 and 2014 in the U.S.^{[4]}

To combat the deadly and addictive effects of oxycodone and hydrocodone, we pursued a vaccine-mediated pharmacokinetic (PK) strategy. In this approach, a small molecule-immunogenic protein conjugate is used to elicit drug-specific IgG antibodies that can bind to freely-circulating opioid molecules and prevent them from entering the CNS to induce MOR activation. When compared to the traditional pharmacologic strategy for treating PO addiction (MOR agonists or antagonists; e.g. methadone, buprenorphine, naltrexone), a PK strategy has several advantages including: (1) an ability to block drug activity without directly interacting with MORs, (2) fewer adverse or dysphoric side-effects, and (3) persistence of circulating antibody for up to a year, using strategically-spaced booster injections.^{[6]} On the whole, such a vaccine could effectively suppress the addiction liability and overdose potential of the target drug over an extended time period without placing excessive compliance demands on the patient.

### SUMMARY

The invention provides, in various embodiments, a hapten for an immunoconjugate, comprising a compound of formula (I)

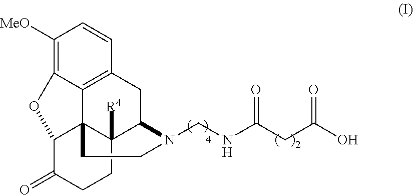

wherein R⁴ is H or OH. An immunoconjugate of formula (II) can be prepared from the hapten of formula (I),

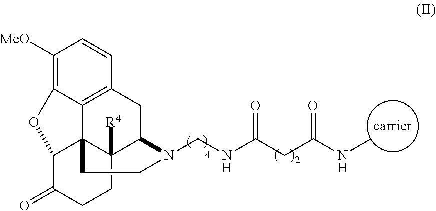

wherein R⁴ is H or OH, and wherein carrier is tetanus toxoid (TT), bovine serum albumin (BSA), or a Dynabead.

When administered to an animal for production of a responsive antibody, the immunoconjugate of formula (II) can raise in the animal an opioid-specific antibody suitable for binding a serum concentration of oxycodone or hydrocodone in a human. The antibody production method comprises administering to the animal an effective amount of the immunoconjugate of formula (II), wherein carrier is tetanus toxoid or bovine serum albumin, then, extracting from the animal the antiserum generated in the immune system of the animal comprising the opioid-specific antibody.

The invention further provides, in various embodiments, a method of forming an antibody-bound opioid drug in a body fluid of a human, wherein the opioid drug is oxycodone or hydrocodone, comprising administering to the human having a serum concentration of the opioid drug, an effective amount or concentration of the opioid-specific antibody, wherein the opioid drug is oxycodone or hydrocodone.

Further, the invention can provide a method of blocking an effect in a human of an opioid drug comprising oxycodone or hydrocodone, comprising administering to the human having a serum concentration of the opioid drug, an effective amount or concentration of the opioid-specific antibody.

Consequently, the invention can further provide a method of reversing an overdose of an opioid drug comprising oxycodone or hydrocodone in a human, comprising administering to the human having a serum concentration of the opioid drug, an effective amount or concentration of the opioid-specific antibody.

Further, the invention can further provide a method of reducing or eliminating a perceived mental effect of an opioid in a human, wherein the human has a serum concentration of an opioid drug such as oxycodone or hydrocodone sufficient to cause a mental effect, comprising administering to the human an effective amount or concentration of the opioid-specific antibody. For example, the perceived mental effect can be euphoria.

### BRIEF DESCRIPTION OF THE FIGURES

FIG. 1 depicts the structures of prescription opioids and heroin.

FIG. 2 shows the timeline of experiments and anti-oxycodone and anti-hydrocodone antibody titers, 50 μg of Oxy-TT and Hydro-TT were formulated with 1 mg of alum and 50 μg of CpG ODN 1826, and administered IP to N=6 mice, respectively. Titers were determined by ELISA against Oxy-BSA and Hydro-BSA conjugate. Key: a=antinociception assay, b=bleed, bb=blood/brain biodistribution study, p=PK study, s=SPR analysis, v=vaccination. Points denote mean±SEM.

FIG. 3 depicts data with respect to behavioral results for intraperitoneally vaccinated and unvaccinated mice (n=6) in the hot plate (A-D) and tail flick (E-H) antinociception assays. Vaccinated and unvaccinated mice were cumulatively dosed with the specified drug and latency to nociception was measured by hot plate and tail flick tests. Bars denote means±SEM. **=p<0.01; ***=p<0.001 two-tailed t-test.

FIG. 4 shows the serum pharmacokinetic results for vaccinated and unvaccinated mice (n=6 overall, n=2 per time point) following intraperitoneal cassette injection of 4 mg/kg oxycodone and hydrocodone. Bars denote means±SEM. Dotted lines represent the 95% Cl for each regression.

FIG. 5 depicts graphical evidence pertaining to the biodistribution of oxycodone and hydrocodone in blood and brain samples. Vaccinated and naive mice (n=3 each) were intraperitoneally dosed with hydrocodone at 8 mg/kg or oxycodone at 4 mg/kg, then trunk blood and brain were harvested 15 min post injection. The drug quantification was performed by LCMS analysis. Bars denote means±SEM. *=p<0.01; ***=p<0.0001 one way ANOVA.

FIG. 6 depicts graphical evidence pertaining to survival rates following administration of lethal doses of prescription opioids. Vaccinated and unvaccinated animals (n=7-8) were dosed SC with 426 mg/kg of oxycodone (A) or 86 mg/kg of hydrocodone (B), and survival was measured continuously for 120 min, then again at 12 and 24 hours post injection.

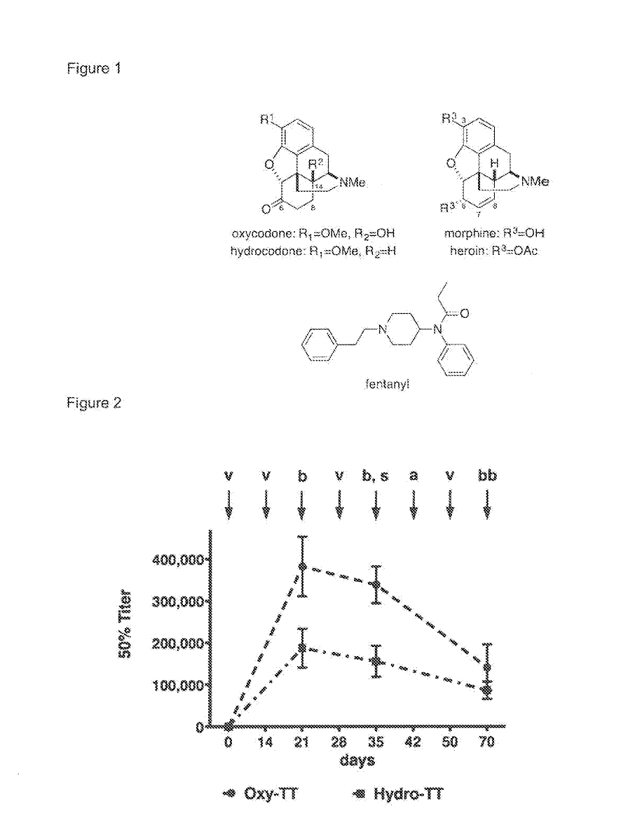

### DETAILED DESCRIPTION

While there have been disclosures of both oxycodone and hydrocodone-targeted conjugate vaccines, the haptens used in these studies did not fully maintain the correct chemical architecture of the drugs being questioned, based on the concept of immunopharmacotherapy.^{[7]} As we have previously made known, haptens that faithfully preserve the original structure of the target drug are often superior in generating high-affinity antibodies to those that do not.^{[8]} Taking this general design principle into account, we chose to replace the N-methyl group of both oxycodone and hydrocodone with a succinic anhydride-derived linker.

Based upon this immunochemical wisdom, syntheses of Oxycodone-TT (Oxy-TT) and Hydrocodone-TT (Hydro-TT) were accomplished as shown in Scheme 1. Importantly, replacement of the N-methyl group of oxycodone and hydrocodone with an N-butyl group possessing a succinamic acid moiety allowed the haptens to be conjugated to tetanus toxoid (TT) under standard amide-coupling conditions (Scheme 1).^{[9]} We selected TT as our carrier protein due to its demonstrated efficacy in vaccines against drugs of abused.^{[10]} Following conjugation, both Oxy-TT and Hydro-TT were formulated with alum and CpG ODN 1826 as adjuvants, which have likewise demonstrated good efficacy in the course of our previous studies.^{[10-11]}

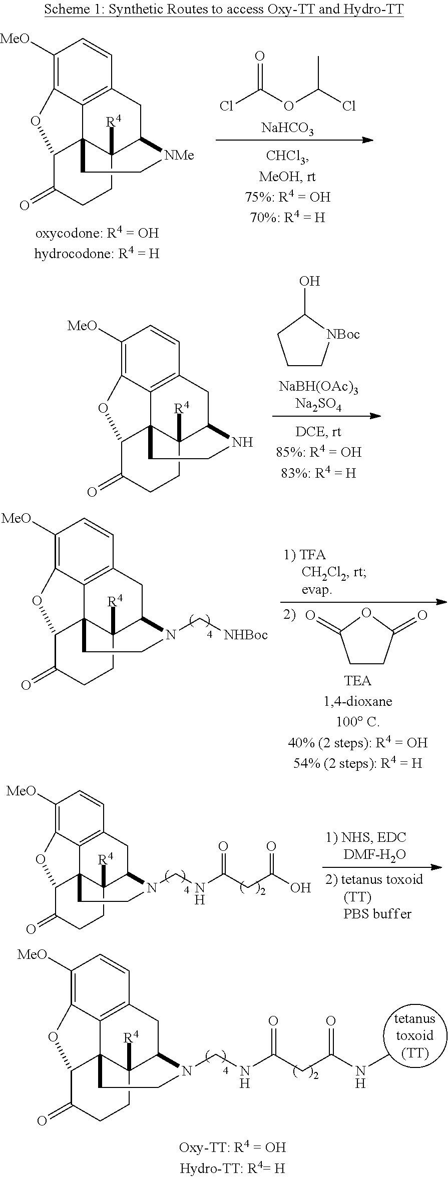

The resulting vaccines were administered to mice over the course of four intraperitoneal (IP) injections. This sequence of events led to both Oxy-TT and Hydro-TT inducing high antibody mid-point titers when tested against their cognate coating antigen (ca. 400,000 for Oxy-TT and 200,000 for Hydro-TT) (FIG. 2). Notably, although these titers dropped somewhat by the end of the study, there was still a substantial antibody presence in both cohorts of animals on day 70.

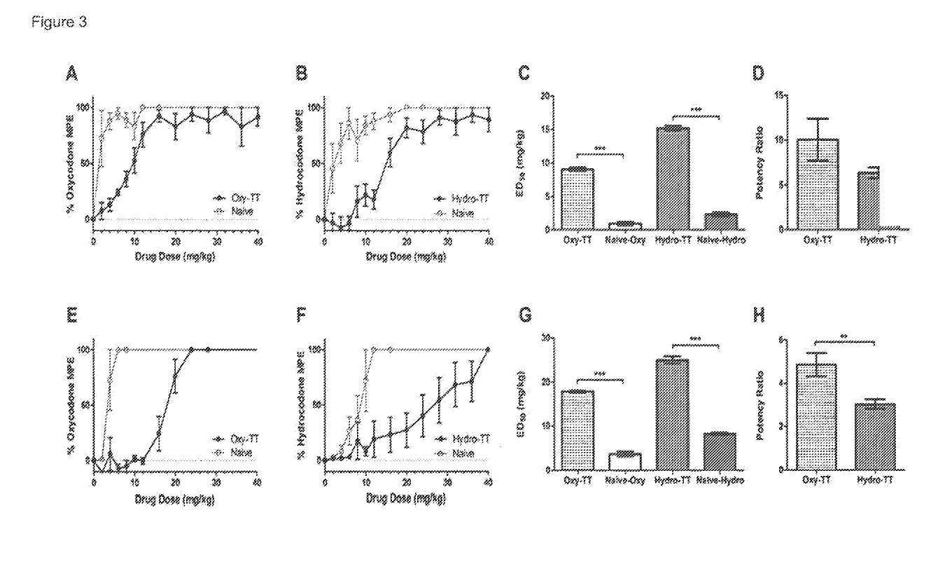

To assess the functional efficacy of our vaccines, we conducted both hot plate and tail flick antinociception assays, which are often used for measuring the supraspinal and spinal analgesic potency of opioid drugs in rodent models.^{[12]} In these assays, oxycodone and hydrocodone raise pain thresholds in a dose-responsive manner, and their efficacy can be quantified by measuring a response latency to the heat stimulus across several doses. Since antibodies from vaccinated mice bind to the target drugs and blunt their pharmacologic effect, an effective vaccine will present a rightward shift in these antinociception assays. In the current study, vaccination resulted in large increases for both oxycodone and hydrocodone ED₅₀s (9.06±0.61 mg by Oxy-TT, 15.17±0.50 mg by Hydro-TT). Quantification of these ED₅₀ shifts as dose ratios, (ca. 10-fold shift in oxycodone ED₅₀, and ca. 6-fold shift in hydrocodone ED₅₀) revealed the significant pharmacodynamic buffering capacity that vaccination was able to achieve (FIG. 3).

In order to further characterize the functional utility of this strategy, we next explored the ability of the resulting antibodies to bind clinically relevant concentrations of the two POs in vitro. A recent clinical study in oxycodone and hydrocodone abusers indicated that a steady state serum concentration of ca. 300 ng/mL was correlated with the subjective perception of feeling high.^{[13]} Thus, in order to judge whether our vaccines could generate a robust antibody response to effectively block this concentration, serum from mice vaccinated with Oxy-TT and Hydro-TT were challenged with 300 ng/mL of oxycodone and hydrocodone. The amount of drug able to diffuse across a dialysis membrane was then quantified by LCMS. Gratifyingly, we found that serum from vaccinated mice could bind all free drug at this concentration, while unvaccinated serum exhibited no specific binding for these species. As anticipated, the antibodies generated by each vaccine did not discriminate between oxycodone and hydrocodone. This latter finding at first glance would appear to run countercurrent with our hypothesis of the need for haptens to be structurally congruent to the drug. However, simple molecular modeling exercises based upon either the Oxy-TT or Hydro-TT hapten show the C-14 position's atom to be spatially sequestered, the outcome of this being an immunochemically silent epitope, which is not distinguished by the immune system.

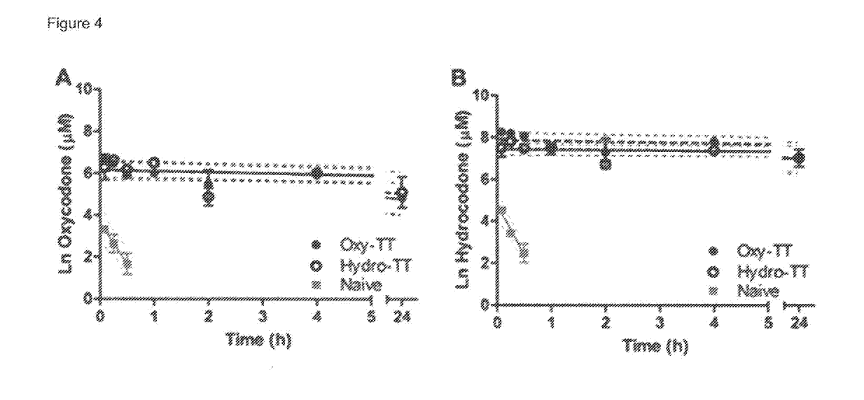

We were next led to consider the vaccines' effect on drug metabolism. To measure the rate of metabolic clearance of oxycodone and hydrocodone, a cassette preparation containing both drugs was administered to vaccinated and unvaccinated mice, followed by serial blood sampling over the course of 24 h.^{[14]} The amount of drug remaining in the blood at each time point was then quantified using LCMS (FIG. 4). In unvaccinated mice, oxycodone and hydrocodone were found to have a serum half-life between 10-20 minutes. To our surprise, vaccination was able to prolong drug half-life to between 12-38.5 hours—a 68 to 268-fold increase over unvaccinated animals. We contrast this to a report by Pfizer, where vaccination against nicotine only generated a 7.4-fold in half-life.^{[14]} Our analysis also indicated that the vaccines were increasing the peak concentration of the drugs in the serum, which is consistent with the expectation that vaccination will sequester free drug in the periphery. As half-life (t_{1/2}) is governed by the relationship t_{1/2}=(0.693*V_{d})/CL, these findings indicate that the vaccines' effect on half-life is likely to be due to a decreased rate of clearance (CL), rather than an increase in the volume of distribution (V_{d}) of the drugs.

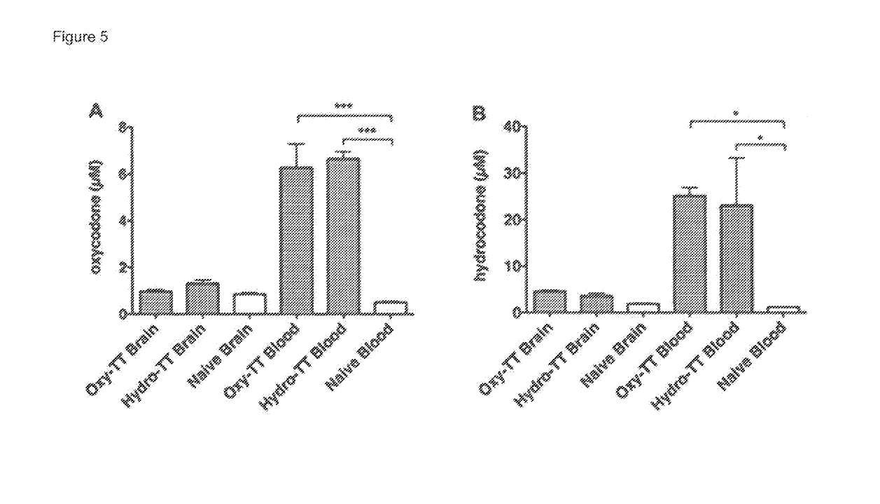

To assess the link between these observed pharmacodynamic and pharmacokinetic effects, we next investigated how the vaccines could alter the bio-distribution of oxycodone and hydrocodone. After treating vaccinated and unvaccinated mice with oxycodone or hydrocodone, the mice were sacrificed at 15 min via rapid decapitation and the drug concentration in blood and brain was measured by LCMS. As with the PK data discussed vide supra, our results from this experiment clearly show that vaccine-induced serum antibodies can effectively trap large amounts of free drug in the blood (FIG. 5). Interestingly, although all mice received the same amount of the drug, the total concentration of drug found in vaccinated mice appeared higher than in unvaccinated animals. When considered together with the in vitro serum binding results vide supra, it is plausible that the significant decrease in the fraction of unbound drug in the bloodstream of vaccinated animals is ultimately limiting the rate of hepatic clearance for the test compounds.

Having established the remarkable pharmacokinetic effects of these two vaccines, we decided to seek a deeper understanding of the vaccines' polyclonal response, investigating their kinetics as a means to further tease out efficacy. We therefore looked to surface plasmon resonance (SPR) (Table 1), where we were able to directly measure the antibody binding kinetics (kₐ: Association rate constant, k_{d}: Dissociation rate constant, K_{D}=k_{d}/kₐ: Dissociation equilibrium constant) for each opioid.[10b] Using this design strategy affinity purified polyclonal immunoglobulin G (pIgG) was loaded on an SPR chip and challenged with free POs. As shewn in Table 1, both anti-oxycodone and anti-hydrocodone pIgGs exhibited extremely high affinity to both cognate drugs (sub-nanomolar K_{D}). As we have stated vide supra, this cross-reactivity is due to the C-14 positon being an immunosilent chemical epitope. On the other hand, the pIgGs possessed lower affinity for morphine, presumably due to its vast structural differences at C-3 (hydroxyl), C-6 (hydroxyl) and C-7,8 (alkene). This interpretation is also supported by the relatively weak K_{D}s for heroin and fentanyl. Additional drug-hapten structural analysis demonstrates why, heroin containing two bulky acetyls and a C-7,8-alkene, and fentanyl lacking the morphinan skeleton result in poor antibody affinity for these drugs. Thus, structurally different analgesics are likely to remain effective therapeutic options for pain management in individuals treated with Oxy-TT or Hydro-TT.

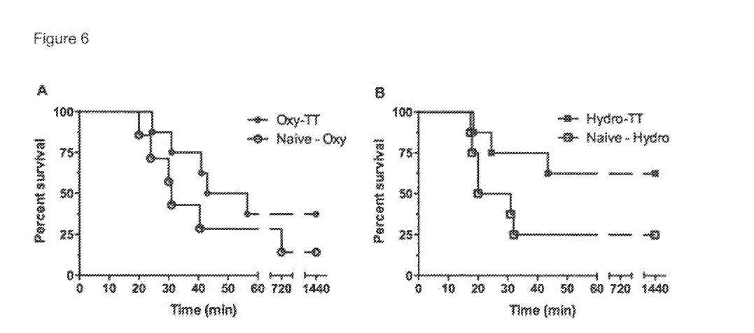

Because induced antibodies exhibited both a prolonged duration of binding and extremely high affinity for free oxycodone and hydrocodone, we assessed the ability of Oxy-TT and Hydro-TT to protect against acute overdose. When unvaccinated mice were dosed subcutaneously (SC) with 426 mg/kg of oxycodone, a 14.2% survival rate was seen at 24 hours, while overall survival was increased to 37.5% in Oxy-TT vaccinated animals. Likewise, in unvaccinated mice given 86 mg/kg of hydrocodone SC, a 25% survival rate was observed at 24 hours, whereas 62.5% of the animals vaccinated with Hydro-TT survived (FIG. 6). We highlight these results as over the past thirty years, vaccination and overdose treatment was only thought to be viable using a large bolus of a monoclonal antibody (passive vaccine). Clearly, while these active vaccines do not provide complete protection, our findings do challenge the scattered skeptics and even naysayers who have stated that active vaccines cannot attenuate drug overdose.

In summary, these opioid conjugate vaccines demonstrated robust production of high-affinity antibodies with ample in vivo neutralization of their cognate opioids' centrally-mediated pharmacodynamic effects, even while dramatically prolonging the half-life of these compounds in the bloodstream. Crucially, the current study also established that Oxy-TT and Hydro-TT vaccines can limit acute mortality from lethal doses of hydrocodone and oxycodone, which could ultimately help to halt the ongoing PO overdose epidemic. Hapten methodologies designed herein are expected to inform future vaccination studies for other classes of frequently abused drugs.

### DOCUMENTS CITED

[1] CDC, National Vital Statistics System, Mortality File. (2015) 2015.

[2] NIDA, Overdose Death Rates (Revised December 2015) 2015.

[3] a) I. Giraudon, K. Lowitz, P. I. Dargan, D. M. Wood, R. C. Dart, Br J Clin Pharmacol 2013, 76, 823-824; b) J. van Amsterdam, W. van den Brink, Curr Drug Abuse Rev 2015. 8. 3-14; c) J. G. del Pozo, A. Carvajal, J. M. Viloria, A. Velasco, V. G. del Pozo, Eur J Clin Pharmacol 2008, 64, 411-415.

[4] CDC, Morbidity and Mortality Weekly Report 2016, 50.

[5] NAHDA, HHS Publication No. (SMA) 13-4760, DAWN Series D-39. Rockville. Md., 2013 2011.

[6] B. A. Martell, E. Mitchell, J. Poling, K. Gonsai, T. R. Kosten, Biol Psychiatry 2005, 58, 158-164.

[7] a) M. Pravetoni, M. Le Naour, T. M. Harmon, A. M. Tucker, P. S. Portoghese, P. R. Pentel, J Pharmacol Exp Ther 2012, 341, 225-232; b) M. Pravetoni, M. Le Naour, A. M. Tucker, T. M. Harmon, T. M. Hawley, P. S. Portoghese, P. R. Pentel, J Med Chem 2013, 56, 915-923; c) M. Pravetoni, M. D. Raleigh, M. Le Naour, A. M. Tucker, T. M. Harmon, J. M. Jones, A. K. Birnbaum, P. S. Portoghese, P. R. Pentel. Vaccine 2012, 30, 4617-4624.

[8] G. N. Stowe, L. F. Vendruscolo, S Edwards, J. E. Schlosburg, K. K. Misra, G. Schulteis, A. V. Mayorov, J. S. Zakhari, G. F. Koob, K. D. Janda, J Med Chem 2011, 54, 5195-5204.

[9] a) H. Kunz, K. von dem Bruch, Methods Enzymol 1994, 247, 3-30; b) H. Kunz, S. Birnbach, Angewandte Chemie-International Edition in English 1986, 25, 360-362; c) H. Kunz, S. Birnbach, P. Wernig, Carbohyd Res 1990, 202, 207-223.

[10] a) N. T. Jacob, J. W. Lockner, J. E. Schlosburg, B. A. Ellis, L. M. Eubanks, K. D. Janda, J Med Chem 2016, 59, 2523-2529; b) P. T. Bremer, A. Kimishima, J. E. Schlosburg, B. Zhou, K. C. Collins, K. D. Janda, Angew Chem Int Ed Engl 2016, 55, 3772-3775.

[11] P. T. Bremer, J. E. Schlosburg, J. M. Lively, K. D. Janda, Mol Pharm 2014, 11, 1075-1080.

[12] A. W. Bannon, A. B. Malmberg, Curr Protoc Neurosci 2007, Chapter 8, Unit 8 9.

[13] T. L. Morton, K. Devarakonda, K. Kostenbader, J. Montgomery, T. Barrett, L. Webster, Pain Med 2015.

[14] M. J. McCluskie, D. M. Evans, N. Zhang, M. Benoit, S. P. McElhiney, M. Unnithan, S. C. DeMarco, B. Clay, C. Huber, A. Deora, J. M. Thorn, D. R. Stead, J. R. Merson, H. L. Davis, Immunopharmacol Immunotoxicol 2016, 38, 184-196.

TABLE 1
The kinetic constants of anti-oxycodone or anti-hydroxycodone plgG interacting with different compounds.

| purified plgG | compound | kₐ (M⁻¹s⁻¹) | k_{d} (s⁻¹) | K_{D} (M) |
|---|---|---|---|---|
| Anti- | oxycodone^{a} | 3.54 × 10⁶ | 1.39 × 10⁻³ | 3.94 × 10⁻¹⁰ |
| oxycodone | hydrocodone^{a} | 3.78 × 10⁶ | 1.59 × 10⁻³ | 4.19 × 10⁻¹⁰ |
| | morphine^{b} | 4.28 × 10² | 4.70 × 10⁻³ | 1.10 × 10⁻⁵ |
| | heroin^{c} | N.D.^{d} | N.D. | N.D. |
| | fentanyl^{c} | N.D. | N.D. | N.D. |
| Anti- | oxycodone^{a} | 1.47 × 10⁶ | 1.22 × 10⁻³ | 8.30 × 10⁻¹⁰ |
| hydrocodone | hydrocodone^{a} | 5.06 × 10⁶ | 1.21 × 10⁻³ | 2.40 × 10⁻¹⁰ |
| | morphine^{b} | 1.05 × 10⁴ | 3.96 × 10⁻³ | 3.78 × 10⁻⁷ |
| | heroin^{c} | N.D. | N.D | N.D. |
| | fentanyl^{c} | N.D. | N.D. | N.D. |

Kinetic analysis was performed on a BIAcore 3000 instrument (GE Healthcare) equipped with a research-grade CM7 sensor chip.
Concentration series: ^{a}= 0, 1, 2, 4, 8, 16, and 32 nM, ^{b}= 0, 1.25, 2.50, 5.00, 10.00, 20.00, and 40.00 μM, ^{c}No significant binding was observed at concentration of 50 μM. ^{d}Kinetics were not determined.

### EXAMPLES

### Chemistry

Nuclear magnetic resonance (¹H NMR (600 MHz), ¹³C NMR (150 MHz)) spectra were determined on a Bruker 600 instrument unless otherwise noted. Chemical shifts for ¹H NMR are reported in parts per million upfield from chloroform (7.26 ppm) or methanol (3.31 ppm) and coupling constants are in hertz (Hz). The following abbreviations are used for spin multiplicity: s=singlet, d=doublet, t=triplet, q=quartet, m=multiplet, br=broad. Chemical shifts for ¹³C NMR were reported in ppm relative to the center line of a triplet at 77.0 ppm for deuterated chloroform or 49.3 ppm for deuterated methanol. Electrospray Ionization (ESI) mass were obtained on a ThermoFinnigan LTQ Ion Trap. Matrix-assisted Laser Desorption/Ionization (MALDI) mass spectra were obtained on an Applied Biosystems DE. Analytical thin layer chromatography (TLC) was performed on Merck pre-coated analytical plates, 0.25 mm thick, silica gel 60 F₂₅₄. Preparative TLC (PTLC) separations were performed on Merck analytical plates (0.50 or 0.25 mm thick) pre-coated with silica gel 60 F₂₅₄.

### Animals

6-8 week old male Swiss Webster mice (n=6/group) were obtained from Taconic Farms (Germantown, N.Y.). Mice were group-housed in an AAALAC-accredited vivarium containing temperature- and humidity-controlled rooms, with mice kept on a reverse light cycle (lights on: 9 PM-9 AM). All experiments were performed during the dark phase, generally between 1 PM-4 PM. General health was monitored by both the scientists and veterinary staff of the Scripps Research Institute, and all studies were performed in compliance with the Scripps Institutional Animal Care and Use Committee, and were in concordance with the National Institutes of Health Guide for the Care and Use of Laboratory Animals. Blood scrum samples were performed using tail-tip amputation (<1 cm) or retro-orbital puncture in order to collect between 100-150 μL whole blood, and samples then centrifuged at 10000 rpm for 10 min to separate serum.

### Hapten Synthesis

### Oxycodone Hapten Synthesis

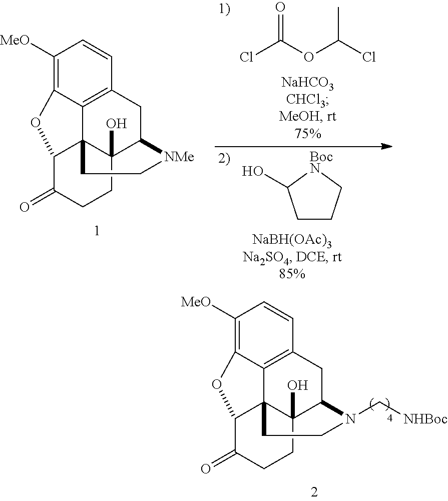

To a solution of 1 (32.3 mg, 102 μmol) in CHCl₃ (1.0 mL) were added NaHCO₃ (86.0 mg, 1.02 mmol) and 1-chloroethyl chloroformate (60.0 μL, 545 μmol) at rt, then the reaction mixture was heated under stirring until reflux. After stirring at reflux for 22 h, additional 1-chloroethyl chloroformate (30.0 μL, 272 μmol) was added to the reaction mixture at rt, then the reaction mixture was again heated under stirring until reflux. After stirring at reflux for additional 3 h, the reaction mixture was allowed to cooled to rt and then treated with MeOH (2.0 mL). After stirring at rt for 6 h, the reaction mixture was quenched with saturated aqueous NH₄Cl and extracted with CH₂Cl₂:MeOH=5:1. The aqueous layer was extracted with CH₂Cl₂:MeOH=5:1 three times. The combined organic extracts were dried over Na₂SO₄, filtered, and evaporated in vacuo. The residual oil was purified by PTLC (SiO₂; CH₂Cl₂:MeOH=5:1) to afford noroxycodone (23.1 mg, 74.8%) as a colorless oil.[1]

To a solution of noroxycodone (23.1 mg, 76.6 μmol) in 1,2-dichloroethane (700 μL) were added Na₂SO₄(32.6 mg, 230 μmol), N-Boc prrolidin-2-ol (43.0 mg, 230 μmol)^{[2]} and NaBH(OAc)₃(25.0 mg, 114 μmol) at rt. After stirring at rt for 1 h, the reaction mixture was quenched with saturated aqueous NaHCO₃ and extracted with CH₂Cl₂:MeOH=9:1. The aqueous layer was extracted with CH₂Cl₂:MeOH=9:1 three times. The combined organic extracts were dried over Na₂SO₄, filtered, and evaporated in vacuo. The residual oil was purified by PTLC (SiO₂; CH₂Cl₂:MeOH=9:1) to afford 2 (30.7 mg, 84.7%) as a colorless oil. [α]_{D}²³-113.1 (c 1.00, CHCl₃); ¹H-NMR (600 MHz, CDCl₃) δ 6.70 (d, J=7.8 Hz, 1H), 6.62 (d, J=7.8 Hz, 1H), 4.46 (s, 1H), 4.56 (or s, 1H), 3.89 (s, 3H), 3.16 (br d, J=4.2 Hz, 2H). 3.09 (d, J=18.0 Hz, 1H). 3.02 (td, J=14.4, 4.8 Hz, 1H), 2.96(br s, 1H), 2.60 (br d, J=17.4 Hz, 2H). 2.53 (br s, 2H), 2.40 (br s, 1H), 2.30 (dt, J=14.4, 3.0 Hz, 1H), 2.16 (br t, J=8.4 Hz, 1H), 2.04 (td, J=17.4, 8.4 Hz, 1H), 1.97 (td. J=12.6, 5.4 Hz, 1H), 1.88 (br t, J=6.6 Hz, 2H), 1.84-1.78 (m, 1H), 1.62 (td, J=15.0, 3.6 Hz, 2H), 1.58 (br dd, J=12.6. 4.2 Hz, 1H), 1.45 (s, 9H); ¹³C-NMR(150 MHz, CDCl₃) δ 208.5, 156.0, 145.0, 143.0, 129.4, 124.9, 119.4, 114.9, 90.3, 70.3, 62.9, 56.6, 53.9, 50.7, 45.9, 43.5, 40.3, 36.1, 31.5, 30.6, 28.4 (3C), 27.9, 24.8, 22.9; HRMS (ESI+) 473.2657 (calcd for C₂₆H₃₇N₂O₆ 473.2652).

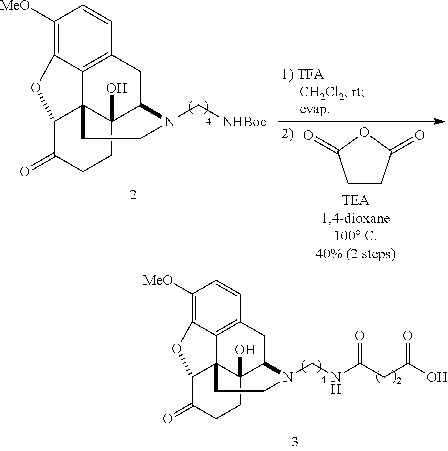

To a solution of 2 (30.7 mg, 64.9 μmol) in CH₂Cl₂ (600 μL) was added TFA (60.0 μL) at rt. After stirring at rt for 2 h, the reaction mixture was co-evaporated with toluene in vacuo. The residual oil was used for the next step without further purification.

To a solution of crude compound in 1,4-dioxane (600 μL) were added TEA (19.5 μL, 140 μmol) and succinic anhydride (6.6 mg, 63.9 μmol) at rt, and then the reaction mixture was heated to 100° C. After stirring at 100° C. for 1 h, the reaction mixture was evaporated in vacuo. The residual solid was purified by PTLC (SiO₂; CH₂Cl₂:MeOH=5:1) to afford 3 (12.3 mg, 40.1% for 2 steps) as a white solid.

[α]_{D}²²-77.1 (c 0.50, CH₃OH); ¹H-NMR (600 MHz. CD₃OD) δ 6.85 (d, J=8.4 Hz, 1H), 6.85 (d, J=8.4 Hz, 1H), 3.89 (s. 3H), 3.65 (d, J=6.0, 1H), 3.40 (ddd, J=13.8, 7.8, 5.4 Hz, 1H), 3.34 (d, J=19.8, 1H), 3.18 (dt, J=13.8, 2.4 Hz, 2H), 3.14 (td, J=13.2, 3.6 Hz, 1H), 3.06 (td, J=14.4, 4.8 Hz, 1H), 3.02 (ddd, J=19.2, 6.0, 1.2 Hz, 1H), 2.97 (ddd, J=12.6, 10.8, 4.8 Hz, 1H), 2.77 (td, J=13.2, 5. Hz, 2H), 2.60 (q, J=4.2 Hz, 1H), 2.57 (dd, J=9.6, 60 Hz, 1H), 2.50 (ddd, J=12.0, 6.6, 4.8 Hz, 1H), 2.46 (dd. J=9.0, 5.4 Hz, 1H), 2.39 (ddd, J=15.0, 7.2, 6.0 Hz, 1H), 2.22 (dt. J=15.0, 3.6 Hz, 1H), 2.06 (ddd, J=13.8, 4.8, 2.4 Hz, 1H), 1.82-1.73 (m. 1H), 1.73-1.62 (m, 1H), 1.63 (td. J=14.4, 3.6 Hz, 1H), 1.62-1.55 (m, 3H); ^{—}C-NMR (150 MHz, CD₃OD) δ 209.7, 180.1, 176.3, 146.7, 145.0, 129.8, 124.7, 121.7, 117.9, 91.2, 71.6, 64.0, 57.9, 54.7, 51.0, 47.6, 38.9, 36.3, 33.7, 33.6, 32.7, 29.8, 27.9, 24.4, 23.0; HRMS (ESI+) 473.2295 (calcd for C₂₅H₃₃N₂O₇ 473.2288).

### Hydrocodone Hapten Synthesis

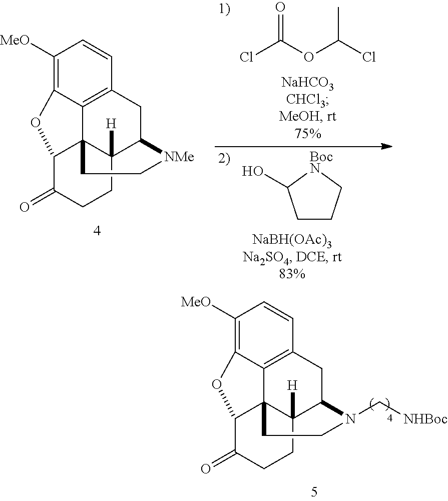

To a solution of 4 (44.8 mg, 150 μmol) in CHCl₃ (400 μL) were added NaHCO₃ (50.0 mg, 595 μmol) and 1-chloroethyl chloroformate (33.0 μL, 297 μmol) at rt, then the reaction mixture was heated under stirring unlit reflux. After stirring at reflux for 4.5 h, the reaction mixture was allowed to cooled to rt and then treated with MeOH (1.0 mL). After stirring at rt for 11 h, the reaction mixture was quenched with saturated aqueous NH₄Cl and extracted with CH₂Cl₂:MeOH=5:1. The aqueous layer was extracted with CH₂Cl₂:MeOH=5:1 three times. The combined organic extracts were dried over Na₂SO₄, filtered, and evaporated in vacuo. The residual oil was purified by PTLC (SiO₂; CH₂Cl₂:MeOH=5:1) to afford norhydrocodone (31.9 mg, 74.7%) as a colorless oil.

To a solution of norhydrocodone (15.6 mg, 54.6 μmol) in 1,2-dichloroethane (500 μL) were added Na₂SO₄(23.2 mg, 163 μmol), N-Boc prrolidin-2-ol (30.6 mg, 163 μmol) and NaBH(OAc)₃(17.8 mg, 81.4 μmol) at rt. After stirring at rt for 2.5 h, the reaction mixture was quenched with saturated aqueous NaHCO₃ and extracted with CH₂Cl₂:MeOH=9:1. The aqueous layer was extracted with CH₂Cl₂:MeOH=9:1 three times. The combined organic extracts were dried over Na₂SO₄, filtered, and evaporated in vacuo. The residual oil was purified by PTLC (SiO₂; CH₂Cl₂:MeOH=9:1) to afford 5 (20.7 mg, 83.0%) as a colorless oil. [α]_{D}²³-86.4 (c 1.00, CHCl₃); ¹H-NMR (600 MHz, CDCl₃) δ 6.69 (d, J=8.4 Hz, 1H), 6.61 (d, J=8.4 Hz, 1H), 5.13 (br s, 1H), 4.64 (s. 1H), 3.25 (br s, 1H), 3.14 (br s, 2H), 2.95 (d, J=18.0 Hz, 1H), 2.64 (br d, J=11.4 Hz, 1H), 2.60 (br d, J=10.8 Hz, 1H), 2.53 (br s, 2H), 2.41 (dt, J=13.8, 3.6 Hz, 1H), 2.37 (br dd, J=13.8, 4.8 Hz, 1H), 2.35-2.30 (m, 1H), 2.15 (br t, J=11.4 Hz, 1H), 2.09 (br t, J=12.6 Hz, 1H), 1.83 (dq, J=12.6, 4.2 Hz, 1H), 1.79 (br d, J=10.8 Hz, 1H), 1.61-1.50 (m, 3H), 1.50-1.38 (m, 4H), 1.45 (s, 9H), 1.24 (qd. J=13.8, 3.6 Hz, 2H); ¹³C-NMR (150 MHz, CDCl₃) δ 207.8, 156.0, 145.4, 142.8, 127.3, 126.1, 119.7, 114.6, 91.3, 57.3, 56.8, 54.3, 47.3, 45.0, 42.3, 40.5, 40.2, 35.3, 28.5 (3C), 28.4 (2C), 27.8, 25.6, 20.8; HRMS (ESI+) 457.2709 (calcd for C₂₆H₃₇N₂O₅ 457.2702).

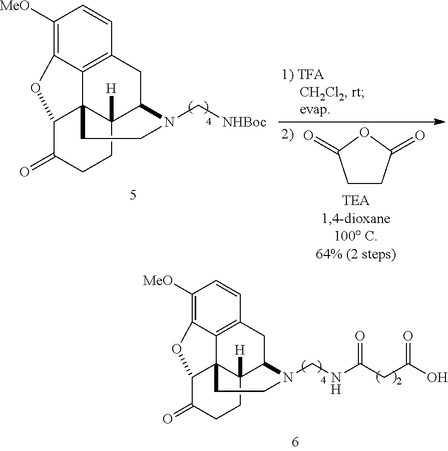

To a solution of 5 (18.6 mg, 407 μmol) in CH₂Cl₂ (400 μL) was added TFA (40.0 μL) at rt. After stirring at rt for 5 h, the reaction mixture was co-evaporated with toluene in vacuo. The residual oil was used for the next step without further purification.

To a solution of crude compound in 1,4-dioxane (400 μL) were added TEA (17.0 μL, 122 μmol) and succinic anhydride (4.1 mg, 39.7 μmol) at rt, and then the reaction mixture was healed to 100° C. After stirring at 100° C. for 2 h, the reaction mixture was evaporated in vacuo. The residual solid was purified by PTLC (SiO₂; CH₂Cl₂:MeOH:MeCN=6:1.5:2) to afford 6 (11.9 mg, 63.9% for 2 steps) as a white solid.

[α]_{D}²²-50.9 (c 0.78, CH₃OH); ¹H-NMR (600 MHz, CD₃OD) δ 6.83 (d, J=7.8 Hz, 1H), 6.77 (d, J=7.8 Hz, 1H), 4.93 (s, 1H), 3.91 (s. 3H), 3.89-3.86 (m, 1H), 3.31-3.29 (m, 1H), 3.20 (br dd, J=13.2, 4.2 Hz, 1H), 3.17 (d, J=19.2 Hz, 1H), 3.09 (dt, J=10.2, 6.6 Hz, 2H), 3.01 (dt, J=12.6, 3.0 Hz, 1H), 2.83 (dd, J=19.8, 6.0 Hz, 2H), 2.64 (td, J=13.8, 4.2 Hz, 1H), 2.60 (td, J=12.6, 3.6 Hz, 1H), 2.55 (t, J=6.0 Hz, 2H), 2.46 (td. J=13.2, 4.8 Hz, 1H), 2.43 (t, J=6.0 Hz, 2H), 2.32 (dt, J=13.8, 3.0 Hz. 1H), 1.98 (dq, J=13.8, 3.6 Hz, 1H), 1.85 (ddd, J=13.8, 1.8, 4.2 Hz, 1H), 1.82-1.74 (m, 2H), 1.66-1.59 (m, 2H), 1.15 (qd, J=12.6, 3.0 Hz, 1H); ¹³C-NMR (150 MHz, CD₃OD) δ 209.8, 180.4, 176.3, 147.1, 144.9, 127.9, 125.4, 121.9, 117.7, 92.2, 59.9, 58.0, 54.6, 47.5, 47.4, 40.5 (2C), 38.3, 34.3, 33.9, 33.6, 27.5, 26.4, 22.5 (2C); HRMS (ESI+) 457.2346 (calcd for C₂₅H₃₃N₂O₆ 457.2339).

### Hapten Conjugation to Bovine Serum Albumin (BSA), Tetanus Toxoid (TT) and Dynabeads®

### Oxycodone Hapten Conjugation to Bovine Serum Albumin (BSA) and Tetanus Toxoid (TT)

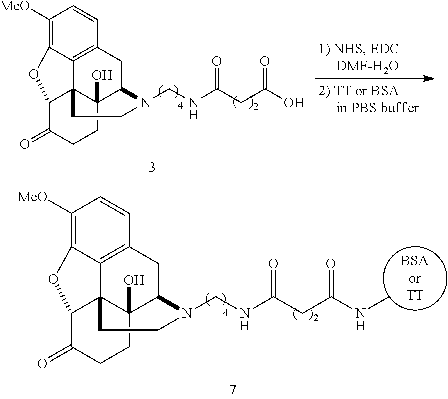

To a solution of 3 (2.6 mg, 5.54 μmol) in DMF (99.0 μL) and H₂O (11.0 μL) were added NHS (N-hydroxysuccinimide) (2.0 mg, 16.8 μmol) and EDC (N-(3-dimethylaminopropyl)-N′-ethylcarbodiimide) (3.0 mg, 15.6 μmol) at rt. After stirring at rt for 5 h, the reaction mixture was divided into two portions.

One portion (66.3 μL) was added into a solution of BSA (Thermo Scientific) in pH 7.4 PBS buffer (73.1 μL, 10.0 mg/mL) at rt, and the other portion (43.7 μL) was added into a solution of TT (UMass Biologics) in pH 7.4 PBS buffer (734 μL, 1.5 mg/mL) at rt. After stirring at rt for 12 h, each of the reaction mixture was dialyzed against pH 7.4 PBS buffer at rt using a Slide-A-Lyzer 10 K MWCO dialysis device. The buffer was exchanged every 2 h for 6 h, and then dialysis was continued for 12 h at 4° C. The conjugates were quantified by BCA assay and stored at 4° C.

### Hydrocodone Hapten Conjugation to Bovine Serum Albumin (BSA) and Tetanus Toxoid (TT)

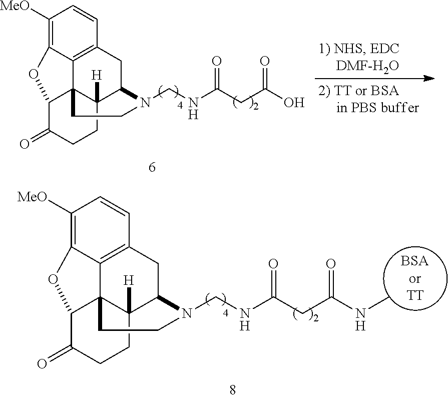

To a solution of 3 (2.1 mg, 4.5 μmol) in DMF (81.0 μL) and H₂O (9.0 μL) were added NHS (N-hydroxysuccinimide) (1.6 mg, 13.4 μmol) and EDC (N-(3-dimethylaminopropyl)-N′-ethylcarbodiimide) (2.5 mg, 13.0 μmol) at rt. After stirring at rt for 5 h, the reaction mixture was divided into two portions.

One portion (42.6 μL) was added into a solution of BSA (Thermo Scientific) in pH 7.4 PBS buffer (50.5 μL, 10.0 mg/mL) at rt, and the other portion (43.8 μL) was added into a solution of TT (UMass Biologics) in pH 7.4 PBS buffer (734 μL, 1.5 mg/mL) at rt. After stirring at rt for 12 h, each of the reaction mixture was dialyzed against pH 7.4 PBS buffer at rt using a Slide-A-Lyzer 10 K MWCO dialysis device. The buffer was exchanged every 2 h for 6 h, and then dialysis was continued for 12 h at 4° C. The conjugates were quantified by BCA assay and stored at 4° C.

### Oxycodone and Hydrocodone Hapten Conjugation to Dynabeads®

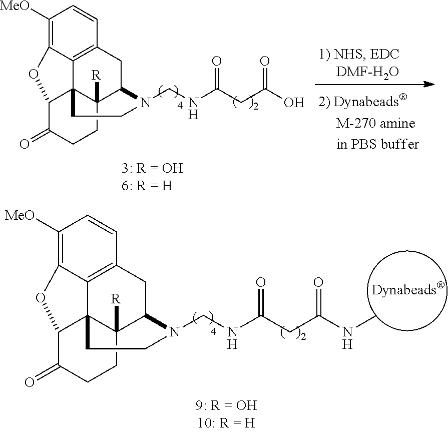

To a solution of 3 or 6 (1.0 mg, 2.1 μmol) in DMF (60.0 μL) and H₂O (7.0 μL) were added NHS (2.4 mg, 20.8 μmol) and EDC (4.0 mg, 208 μmol) at rt. After stirring at rt for 12 h, a 60 μL aliquot of the reaction mixture was added into 1.0 mL of Dynabeads® M-270 Amine (washed 4 times with pH 7.4 PBS buffer prior to use) in pH 7.4 PBS buffer at rt. After stirring at rt for 2 h, the beads were washed with pH 7.4 PBS buffer for two times and stored at 4° C.

### Vaccine Formulation and Administration

On a per mouse basis, 50 μg Oxy-TT or Hydro-TT+50 μg CpG ODN 1826 (Eurofins) in about 60 μl pH 7.4 PBS buffer was combined with 100 μl (10 mg/mL) of Alum (Invivogen) and vortex mixed for 30 min. The suspension was injected intraperitoneally to a mouse (6 mice per group) at weeks 0, 14, 28 and 50. Each mouse was bled at week 21, 35 and 70.

### MALDI-ToF Analysis

In order to quantify copy number (hapten density) for each hapten BSA conjugates prepared in this study, samples were submitted for MALDI-TOF analysis and compared MW of Oxy-BSA and Hydro-BSA with MW of unmodified BSA as per the formula (BSA was used as a surrogate for TT):

copy number=(MWₕₐₚₜₑₙ₋ₚᵣₒₜₑᵢₙ−MWₚᵣₒₜₑᵢₙ)/(MWₕₐₚₜₑₙ−MW_{water})

MW_{BSA}=66,500 Da, MW_{water}=18 Da
MW_{oxycodone hapten}=472.5 Da, MW_{Oxy-BSA}=77,852 Da,
copy number_{Oxy-BSA}=25.0
MW_{hydrocodone hapten}=456.5 Da, MW_{Hydro-BSA}=79.895 Da
copy number_{Hydro-BSA}=30.5

### ELISA Procedure

First, half-area high-binding 96-well microliter plates (Costar 3690) were coated with 25 μg of Oxy-BSA or Hydro-BSA per well overnight at 37° C., allowing the liquid to evaporate. Following blocking with 5% skim milk in pH 7.4 PBS (Fisher Scientific) buffer for 1 h at rt, vaccinated mouse serum was serially diluted 1:3 in 2% BSA in pH 7.4 PBS buffer across the 12 columns starting at 1:1000. After a 2 h incubation at rt, the plates were washed 10 times with H₂O, and donkey anti-mouse IgG horseradish peroxidase (HRP) secondary (Jackson ImmunoResearch) at a 1:10,000 dilution in 2% BSA in pH 7.4 PBS buffer was added and incubated for 1.5 h at rt. After washing with H₂O 10 times, 3,3′,5,5′-tetramethylbenzidine (TMB) substrate (Thermo Pierce) was added. 5 min after TMB addition, the mixture was treated with 2 M H₂SO₄. Plates were allowed to incubate 15 min before their absorbances were read at 450 nm. In GraphPad PRISM, absorbance values were normalized to the highest absorbance value per sample, and a curve was fit using the log(inhibitor) vs. normalized response—variable slope equation to determine the midpoint titer and standard errors. Non-vaccinate mice did not contain any detectable anti-Oxy or anti-Hydro titers.

Competitive ELISA was also performed in a similar manner but with an added step: serum at the IC₈₀ dilution was incubated with free oxycodone or hydrocodone dilutions of 1 mM to 0.02 nM (eleven 5-fold dilutions) in Oxy-BSA or Hydro-BSA coated plates for 2 h. The IC₅₀ₛ were calculated to be Oxy-TT against oxycodone: 0.34±0.03 μM, Oxy-TT against hydrocodone: 0.22±0.02, Hydro-TT against hydrocodone: 0.14±0.01, Hydro-TT against oxycodone: 0.18±0.02 μM, Those values are off by a factor of about 70 compared to SPR results.

### Antinociceptive Potency Testing

At least 2 days following a bleed, mice were tested for cumulative oxycodone and hydrocodone response in primarily supraspinal (hot plate) and spinal (tail flick) behavioral tests as previously described.^{[3]} The hot plate test was measured by placing the mouse in an acrylic cylinder (14 cm diameter * 22 cm) on a 54° C. surface and timing latency to perform one of the following nociceptive responses: licking of hindpaw, shaking/withdrawal of hindpaw, or jumping. Typical baseline latency was between 10 to 20 s and a 35 s cutoff was imposed to prevent tissue damage; after response mice were removed from the plate. The tail immersion test was administered by lightly restraining mice in a small pouch constructed from absorbent laboratory underpads and dipping 1 cm of the tip of the tail into a heated water bath, with the time to withdrawal timed. Typical baseline response was 0.5-1 s and a cutoff of 10 s was used to prevent tissue damage. Since tail immersion is a more reflexive behavior, testing order was always hot plate first followed by tail immersion. Immediately following completion of both antinociceptive assays, oxycodone or hydrocodone (2.0 mL/kg in normal saline) was immediately injected intraperitoneally. Testing was repeated roughly 15 min following each injection, and this cycle of testing and injections was repeated with increasing cumulative dosing until full antinociception (i.e. cutoff times surpassed) was observed in both assays.

### Procedures for Serum Binding Study

Mouse sera collected on day 70 were pooled and diluted 10-fold into PBS, using 10 μL of serum per mouse. 100 μL of this diluted serum was placed into one side of a 5 kDa MWCO 6-well Equilibrium Dialyzer (Harvard Apparatus) (n=2). 100 μL of 300 ng/mL of oxycodone or hydrocodone in 1% BSA in PBS was added to the other side of the dialysis membrane.^{[4]} After 22-24 h of equilibration at room temperature on a plate rotator (Harvard Apparatus), a 50 μL sample from each chamber was added to 50 μL MeCN containing 10 μM of d6-hydrocodone or d6-oxycodone as an internal standard (IS). The samples were then centrifuged at 10,000 rpm for 10 min, and 80 μL of supernatant were removed for LCMS analysis. A 5 μL aliquot of each sample was injected into an LCMS system equipped with an Agilent Poroshell 120 SB-C8 column using H₂O+0.1% formic acid and MeCN+0.1% formic acid as the mobile phases. The percentage of MeCN+0.1% formic acid was linearly increased from 5-95% over a 10 minute run (150 μL/min), followed by a 10 minute wash phase at 5% MeCN+0.1% formic acid.

Deuterated and non-deuterated masses were extracted in MassHunter and resulting peaks were integrated. Non-deuterated peak sizes were normalized alongside the IS peak to account for variability across sample runs. The percent of drug bound was calculated using the equation [Serum Counts/(Serum Counts+Buffer Counts)]×100 (Table 2).

TABLE 2
Percent serum binding of 300 ng/ml oxycodone or hydrocodone
via equilibrium dialysis. Data are presented
as the mean ± SEM for two independent replicates.

| | % of Total Drug Bound in the Serum | |
|---|---|---|
| Group (n = 6) | Oxycodone | Hydrocodone |
| Naive | 55 ± 2 | 50 ± 1 |
| Oxy-TT | 100 ± 0 | 100 ± 0 |
| Hydro-TT | 100 ± 0 | 100 ± 0 |

### Procedures for Pharmacokinetic Study

On day 63, three groups (Oxy-TT, Hydro-TT, and Naïve) of male Swiss Webster mice (n=5-6) were injected intraperitoneally (5 ml/kg) with a solution containing 0.8 mg/ml of oxycodone and hydrocodone to provide a dose of 4 mg/kg for each of these drugs. The animals wee then returned to their home cage. Each animal then had two or three blood samples taken via retro-orbital bleed at independent, pre-set periods of time (5, 15, 30, 60, 120, 240, or 1440 minutes), to generate n=1-2 data for each measured time point. The exact time points taken for each mouse were balanced with respect to group. Collected blood samples were stored on ice for 0.5-4 hours, then centrifuged at 10,000 rpm for 10 minutes. The serum was then collected and stored at −20° C. until analysis by LCMS. On the day of LCMS analysis, 20 μL from each sample were added to 80 μL of MeCN containing 1 μM concentrations of d6-hydrocodone and d6-oxycodone. These samples were centrifuged at 10,000 rpm for 10 minutes, and 80 μL of supernatant were collected. Each sample then run on the LCMS using the same injection and run protocol described above. Quantification of the serum concentrations for each drug was achieved through the use of a 10-point standard curve for both hydrocodone and oxycodone, using blank mouse serum that had been spiked with known concentrations (0 nM-10 μM) of each compound. The elimination rate constant (kₑ) for each condition was calculated by plotting the natural log of the concentration value over time during the terminal elimination phase, and finding the slope of the resulting line. The half-life was then calculated for each condition using the formula Ln(2)/kₑ (Table 3).^{[5]}

TABLE 3
Calculated average pharmacokinetic parameters for vaccinated and unvaccinated mice
following intraperitoneal cassette injection of 4 mg/kg oxycodone and hydrocodone.

| | Oxycodone | | | Hydrocodone | | |
|---|---|---|---|---|---|---|
| Group | t_{1/2} | Cₘₐₓ | AUC₀₋₂₄ | t_{1/2} | Cₘₐₓ | AUC₀₋₂₄ |
| (n = 6) | (h) | (ng/mL) | (ng/mL*h) | (h) | (ng/mL) | (ng/mL*h) |
| Oxy-TT | 12.0 | 753.7 | 6455.0 | 19.3 | 3836.3 | 45047.0 |
| Hydro-TT | 15.0 | 750.3 | 7654.0 | 38.5 | 2467.6 | 34161.0 |
| Naïve | 0.2 | 30.9 | 8.9 | 0.1 | 92.1 | 22.8 |

### Procedure for Blood/Brain Biodistribution Study

On day 77, three groups (Oxy-TT, Hydro-TT, and Naïve) of male Swiss Webster mice (n=5-6) were injected intraperitoneally (10 ml/kg) with oxycodone at 0.4 mg/ml or hydrocodone at 0.8 mg/ml, then returned to their home cage. 15 min following injection, the animals were fully anesthetized using nose cones constructed from 50 mL Falcon® conical centrifuge tube (Corning, NY) containing gauze pads soaked in isoflurane. The animals were then rapidly decapitated using a sharp guillotine, the brains were extracted with gongeurs, and trunk blood was collected. The blood was placed on ice for 0.5-2 hours, then centrifuged at 10,000 RPM for 10 min, and the serum was collected. The brain tissue was immediately flash frozen using dry ice cooled acetone bath. Serum and brain samples were stored at −80° C. until extraction and LCMS analysis. To each whole frozen brain, 0.2 M aqueous perchloric acid (1.0 mL) was added. The mixture was homogenized using Bullet Blender® (Next Advance, NY) and then centrifuged at 3000 rpm for 10 min. To a 200 μL aliquot of a supernatant liquid of homogenized brain mixture was added 200 μL (250 ng/ml) of d6-hydrocodone or d6-oxycodone in MeOH and Vortex® mixed for 1 min to equilibrate. The mixture was then extracted with Oasis® PRiME HLB Extraction Cartridge, and the extracted solution was evaporated using GENEVAC®. To each 50 μL aliquot of serum, 0.1 M aqueous perchloric acid (100 μL) and d6-hydrocodone or d6-oxycodone were added, and then the mixture was Vortex® mixed for 1 min to equilibrate. After the equilibration, Solid-phase extraction with Oasis® PRiME HLB Extraction Cartridge was conducted. The extracted solution was then evaporated using GENEVAC®. These tissue samples were analyzed using the injection and run protocol described above, and quantified using a 10-point standard curve for oxycodone and hydrocodone.

### Overdose Study

Four groups (Oxy-TT, Hydro-TT, Oxy-Naïve and Hydro-Naïve) of male Swiss Webster mice (N=7-8) were injected subcutaneously with 426 mg/kg oxycodone or 86 mg/kg hydrocodone in normal saline. Animals were then under constant observation for 2 h, followed by single measurements at 12 h and 24 h after injections. At 24 hours, the following number of animals had died: Oxy-TT, 5/8; Hydro-TT, 3/8; Oxy-Naïve, 6/7; Hydro-Naïve, 6/8.

### Statistics

Statistical analysis was performed in GraphPad Prism 6 (La Jolla, Calif.). All values are reported as means±SEM. Antinociceptive data was transformed from time to % maximum possible effect (% MPE), which is calculated as: % MPE=(test−baseline)/(cutoff—baseline)* 100. This data was then fit using a log(agonist) vs. normalized response non-linear regression. ED₅₀ values and 95% confidence intervals of the ED₅₀ were calculated for each pain test and individual treatment group to determine potency ratios, as well as for verification of statistically significant differences (α<0.05).

Determination of Binding Kinetics of Purified Mouse anti-oxycodone and Anti-hydrocodone Immunoglobulins

The binding kinetics between mouse IGs (purified directly from day 35 bleed using magnetic oxycodone- or hydrocodone-coupled Dynabeads®) and opioids, including oxycodone, hydrocodone, oxymorphone, hydromorphone, morphine, heroin and fentanyl, were determined by surface plasmon resonance using a BIAcore 3000 instrument (GE Healthcare) equipped with a research-grade CM7 sensor chip. The ligand, mouse anti-oxycodone and anti-hydrocodone IgGs (˜150 kDa), were immobilized directly to the CM7 chip surface using NHS/EDC coupling reaction according to manufacturer's instruction. Two additional flow cells served as reference flow cells were also activated by NHS/EDC chemistry. All the surfaces were blocked with a 7 min injection of 1.0 M ethanolamine-HCl (pH 8.5). To collect kinetic data, the analyte, series of concentration of opioids (see Table 1) prepared in HBS-EP+buffer (10 mM HEPES, 150 mM NaCl, 0.05% P20 (pH 7.4)), was injected over the two flow cells at a flow rate of 30 μL/min at a temperature of 25° C. The complex was allowed to associate and dissociate for 300 and 600 s, respectively. Duplicate injections (in random order) of each analyte sample and blank buffer injections were flowed over the two surfaces. The surface was regenerated in running buffer for an extra 1800 s after each analyte injection. Data were collected, double referenced, and were fit to a 1.1 Langmuir interaction model using the global data analysis by BIAevaluation software (ver. 4.1).

### DOCUMENTS CITED IN EXAMPLES SECTION

[1] O. H. Kvemenes, A. M. Nygárd, A. Heggelund, H. Halvorsen, WO2007137782 A1 2007.

[2] Y. Yoshitomi, H. Arai, K. Makino, Y. Hamada, Tetrahedron 2008, 64, 11568-11579.

[3] P. T. Bremer, J. E. Schlosburg, J. M. Lively, K. D. Janda, Mol Pharm 2014, 11,1075-1080.

[4] T. L Morton, K. Devarakonda, K. Kostenbader, J. Montgomery, T. Barrett, L. Webster, Pain Med 2015.

[5] M. J. McCluskie, D. M. Evans, N. Zhang, M. Benoit, S. P. McElhiney, M. Unnithan, S. C. DeMarco, B. Clay, C. Huber, A. Deora, J. M. Thorn, D. R. Stead, J. R. Merson, H. L. Davis, Immunopharmacol Immunotoxicol 2016, 38, 184-196.

All patents and publications referred to herein are incorporated by reference herein to the same extent as if each individual publication was specifically and individually indicated to be incorporated by reference in its entirety.

The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should he understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A hapten for an immunoconjugate, comprising a compound of formula (I) 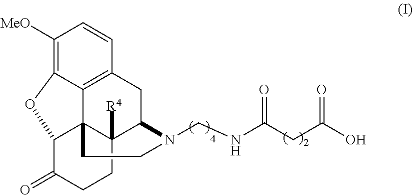
wherein R⁴ is H or OH.

2. An immunoconjugate of formula (II) 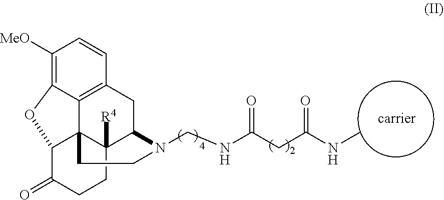
wherein R⁴ is H or OH, and wherein carrier is tetanus toxoid (TT), bovine serum albumin (BSA), or a Dynabead.

3. A method of raising an opioid-specific antibody in an animal, wherein the opioid drug is oxycodone or hydrocodone, comprising administering to the animal an effective amount of the immunoconjugate of formula (II) of claim 2, wherein carrier is tetanus toxoid or bovine serum albumin, then, extracting from the animal the antiserum generated in the immune system of the animal comprising the opioid-specific antibody.

4. A method of forming an antibody-bound opioid drug in a body fluid of a human, comprising administering to the human having a serum concentration of the opioid drug, an effective amount or concentration of the opioid-specific antibody of claim 3, wherein the opioid drug is oxycodone or hydrocodone.

5. A method of blocking an effect in a human of an opioid drug comprising oxycodone or hydrocodone, comprising administering to the human having a serum concentration of the opioid drug, an effective amount or concentration of the opioid-specific antibody of claim 3.

6. The method of claim 5, comprising reversing an overdose of an opioid drug comprising oxycodone or hydrocodone in a human, comprising administering to the human having a serum concentration of the opioid drug, an effective amount or concentration of the opioid-specific antibody of claim 3.

7. The method of claim 5, comprising reducing or eliminating a perceived mental effect of an opioid in a human, wherein the human has a serum concentration of an opioid drug such as oxycodone or hydrocodone sufficient to cause perception of a mental effect, comprising administering to the human an effective amount or concentration of the opioid-specific antibody of claim 3.
